(19) United States
(12) Patent Application Publication
(10) Pub. No.: US 2011/0190540 A1
(43) Pub. Date: Aug. 4, 2011

(54) Methods for Preparing S1P Receptor Agonists and Antagonists

(75) Inventors: Shashank Shekhar, Highland Park, IL (US); Su Yu, Lake Bluff, IL (US); Anthony R. Haight, Wadsworth, IL (US); Preston E. Chmura, Indian Head Park, IL (US); Vimal Kishore, Mundelein, IL (US)

(73) Assignee: ABBOTT LABORATORIES, Abbott Park, IL (US)

(21) Appl. No.: 12/889,513

(22) Filed: Sep. 24, 2010

Related U.S. Application Data
(63) Continuation of application No. 12/702,859, filed on Feb. 9, 2010, now abandoned.
(60) Provisional application No. 61/207,302, filed on Feb. 10, 2009.

(51) Int. Cl. C07C 209/78; C07C 209/74; C07C 209/70

(52) U.S. Cl. 564/393; 564/412; 564/443

(57) ABSTRACT

Disclosed herein are methods of making compounds which are agonists or antagonists of one or more of the individual receptors of the S1P receptor family.

### RELATED APPLICATION

This application is a continuation application claiming priority to U.S. application Ser. No. 12/702,859, filed Feb. 9, 2010, which is a non-provisional application that claims priority to U.S. Provisional Application Ser. No. 61/207,302 filed on Feb. 10, 2009, the contents of which are incorporated herein.

### BACKGROUND

Sphingosine-1-phosphate (S1P) is part of the sphingomyelin biosynthetic pathway and is known to affect multiple biological processes. S1P is formed through phosphorylation of sphingosine by sphingosine kinases (SK1 and SK2), and it is degraded through cleavage by sphingosine lyase to form palmitaldehyde and phosphoethanolamine or through dephosphorylation by phospholipid phosphatases. S1P is present at high levels (about 500 nM) in serum, and it is found in most tissues. S1P can be synthesized in a wide variety of cells in response to several stimuli, which include cytokines, growth factors and G protein-coupled receptor (GPCR) ligands. The GPCRs that bind S1P (currently known as the S1P receptors S1P₁₋₅), couple through pertusis toxin sensitive (Gi) pathways as well as pertusis toxin insensitive pathways to stimulate a variety of processes. The individual receptors of the S1P family are both tissue and response specific and, therefore, are attractive as therapeutic targets.

S1P evokes many responses from cells and tissues. In particular, S1P has been shown to be an agonist at all five GPCRs, S1P₁ (Edg-1), S1P₂ (Edg-5), S1P₃ (Edg-3), S1P₄ (Edg-6) and S1P₅ (Edg-8). The action of S1P at the S1P receptors has been linked to resistance to apoptosis, changes in cellular morphology, cell migration, growth, differentiation, cell division, angiogenesis, oligodendrocyte differentiation and survival, modulation of axon potentials, and modulation of the immune system via alterations of lymphocyte trafficking. Therefore, S1P receptors are therapeutic targets for the treatment of, for example, neoplastic diseases, diseases of the central and peripheral nervous system, autoimmune disorders and tissue rejection in transplantation. These receptors also share 50-55% amino acid identity with three other lysophospholipid receptors, LPA1, LPA2, and LPA3, of the structurally related lysophosphatidic acid (LPA).

GPCRs are excellent drug targets with numerous examples of marketed drugs across multiple disease areas. GPCRs are cell-surface receptors that bind hormones on the extracellular surface of the cell and transduce a signal across the cellular membrane to the inside of the cell. The internal signal is amplified through interaction with G proteins, which in turn interact with various second messenger pathways. This transduction pathway is manifested in downstream cellular responses that include cytoskeletal changes, cell motility, proliferation, apoptosis, secretion and regulation of protein expression, to name a few. S1P receptors make good drug targets because individual receptors are expressed in different tissues and signal through different pathways, making the individual receptors both tissue and response specific. Tissue specificity of the S1P receptors is desirable because development of an agonist or antagonist selective for one receptor localizes the cellular response to tissues containing that receptor, limiting unwanted side effects. Response specificity of the S1P receptors is also of importance because it allows for the development of agonists or antagonists that initiate or suppress certain cellular responses without affecting other responses. For example, the response specificity of the S1P receptors could allow for an S1P mimetic that initiates platelet aggregation without affecting cell morphology.

The physiologic implications of stimulating individual S1P receptors are largely unknown due in part to a lack of receptor type selective ligands. Isolation and characterization of S1P analogs that have potent agonist or antagonist activity for S1P receptors have been limited.

S1P₁ for example is widely expressed, and the knockout causes embryonic lethality due to large vessel rupture. Adoptive cell transfer experiments using lymphocytes from S1P₁ knockout mice have shown that S1P₁ deficient lymphocytes sequester to secondary lymph organs. Conversely, T cells overexpressing S1P₁ partition preferentially into the blood compartment rather than secondary lymph organs. These experiments provide evidence that S1P₁ is the main sphingosine receptor involved in lymphocyte homing and trafficking to secondary lymphoid compartments.

Currently, there is a need for novel, potent, and selective agents, which are agonists or antagonists of the individual receptors of the S1P receptor family, and methods of making the same, in order to address unmet medical needs associated with agonism or antagonism of the individual receptors of the S1P receptor family.

### SUMMARY

The present invention is directed in part to methods of making compounds which are agonists or antagonists of one or more of the individual receptors of the S1P receptor family.

One aspect of the invention relates to a method of making a compound of formula I or a salt thereof,

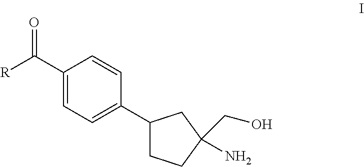

comprising the step of combining a compound of formula II or a salt thereof,

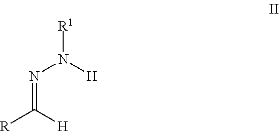

a compound of formula III or a salt thereof,

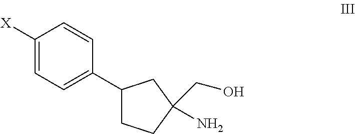

a metal catalyst, a base, and an organic solvent; wherein,

R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;

R¹ is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;

X is halogen or sulfonate; and

the molar ratio of base to the compound of formula III is greater than or equal to about 2.

Another aspect of the invention relates to a method of extracting (1-amino-3-phenylcyclopentyl)methanol from a mixture comprising (1-amino-3-phenylcyclopentyl)methanol and a compound of formula I, or a salt thereof, as defined above, in organic solvent, comprising the step of contacting the mixture with aqueous potassium carbonate having a pH of between about 9 and about 9.5, thereby extracting (1-amino-3-phenylcyclopentyl)methanol from the mixture.

Another aspect of the invention relates to a method of preparing the (R)-mandelic salt of a compound of formula I, as defined above, comprising the step of combining (R)-mandelic acid and a compound of formula I, or a salt thereof, in an organic solvent, thereby forming the (R)-mandelic salt of a compound of formula I.

Another aspect of the invention relates to a method of making a compound of formula IV or a salt thereof:

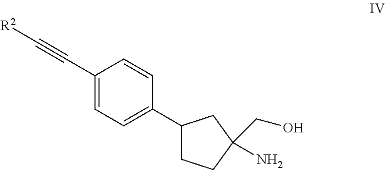

comprising the step of combining a compound of formula III or a salt thereof, as defined above, a compound of formula V:

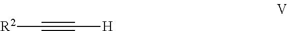

a metal catalyst, and an organic solvent; wherein,

R² is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

Another aspect of the invention relates to a method of making a compound of formula III or a salt thereof, as defined above, comprising the step of combining a compound of formula VI or a salt thereof:

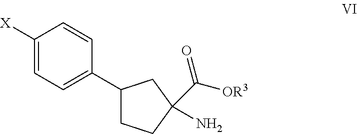

and a reducing agent; wherein,

X is halogen or sulfonate; and

R³ is alkyl.

Another aspect of the invention relates to a method of making a compound of formula IA or a salt thereof,

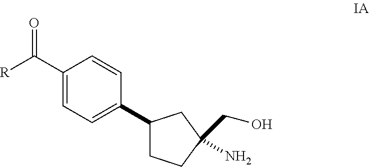

comprising the step of combining a compound of formula II or a salt thereof,

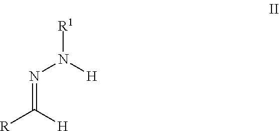

a compound of formula III or a salt thereof,

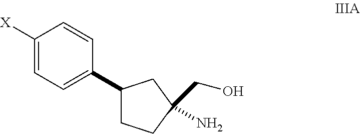

a metal catalyst, a base, and an organic solvent; wherein,

R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;

R¹ is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;

X is halogen or sulfonate; and

the molar ratio of base to the compound of formula IIIA is greater than or equal to about 2.

Another aspect of the invention relates to a method of extracting ((1R,3R)-1-amino-3-phenylcyclopentyl)methanol from a mixture comprising ((1R,3R)-1-amino-3-phenylcyclopentyl)methanol and a compound of formula IA, or a salt thereof, as defined above, in organic solvent, comprising the step of contacting the mixture with aqueous potassium carbonate having a pH of between about 9 and about 9.5, thereby extracting ((1R,3R)-1-amino-3-phenylcyclopentyl)methanol from the mixture.

Another aspect of the invention relates to a method of preparing the (R)-mandelic salt of a compound of formula IA, or a salt thereof, as defined above, comprising the step of combining (R)-mandelic acid with a compound of formula IA, or a salt thereof, in an organic solvent, thereby forming the (R)-mandelic salt of the compound of formula IA.

Another aspect of the invention relates to a method of making a compound of formula IVA or a salt thereof:

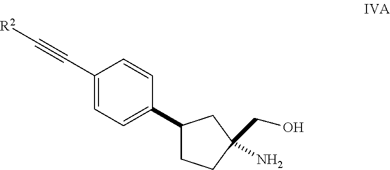

comprising the step of combining a compound of formula IIIA or a salt thereof, as defined above, a compound of formula V:

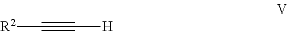

a metal catalyst, and an organic solvent; wherein,

R² is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

Another aspect of the invention relates to a method of making a compound of formula IIIA or a salt thereof, as defined above, comprising the step of combining a compound of formula VIA or a salt thereof:

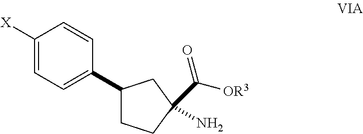

and a reducing agent; wherein,

X is halogen or sulfonate; and

R³ is alkyl.

In certain embodiments, the present invention relates to any one of the aforementioned methods, wherein R is aralkyl.

In certain embodiments, the present invention relates to any one of the aforementioned methods, wherein R is —CH₂CH₂CH₂CH₂Ph.

In certain embodiments, the present invention relates to any one of the aforementioned methods, wherein R¹ is alkyl, substituted alkyl, aryl or heteroaryl.

In certain embodiments, the present invention relates to any one of the aforementioned methods, wherein R¹ is alkyl.

In certain embodiments, the present invention relates to any one of the aforementioned methods, wherein R¹ is —C(CH₃)₃.

In certain embodiments, the present invention relates to any one of the aforementioned methods, wherein X is —Br, —Cl or —I.

In certain embodiments, the present invention relates to any one of the aforementioned methods, wherein X is —Br.

In certain embodiments, the present invention relates to any one of the aforementioned methods, wherein the metal catalyst comprises palladium.

In certain embodiments, the present invention relates to any one of the aforementioned methods, wherein the metal catalyst comprises a bisphosphine ligand.

In certain embodiments, the present invention relates to any one of the aforementioned methods, wherein the metal catalyst comprises a bis(diphenylphosphinophenyl)ether (DPEPhos) ligand.

In certain embodiments, the present invention relates to any one of the aforementioned methods, wherein the metal catalyst is (DPEPhos)PdCl₂.

In certain embodiments, the present invention relates to any one of the aforementioned methods, wherein the metal catalyst is PdCl₂(PPh₃)₂.

In certain embodiments, the present invention relates to any one of the aforementioned methods, wherein the base is a bis(trialkylsilyl)amide salt.

In certain embodiments, the present invention relates to any one of the aforementioned methods, wherein the base is LiN(SiMe₃)₂.

In certain embodiments, the present invention relates to any one of the aforementioned methods, wherein the molar ratio of base to the compound of formula III is about 3.

In certain embodiments, the present invention relates to any one of the aforementioned methods, wherein the molar ratio of base to the compound of formula III is about 4.

In certain embodiments, the present invention relates to any one of the aforementioned methods, wherein the solvent is 1,4-dioxane or dimethoxyethane.

In certain embodiments, the present invention relates to any one of the aforementioned methods, wherein R² is alkoxy-substituted alkyl.

In certain embodiments, the present invention relates to any one of the aforementioned methods, wherein R² is —CH₂CH₂CH₂CH₂CH₂OCH₃.

In certain embodiments, the present invention relates to any one of the aforementioned methods, wherein R³ is —CH₃, —CH₂CH₃ or —CH₂CH₂CH₃.

In certain embodiments, the present invention relates to any one of the aforementioned methods, wherein R³ is —CH₃.

### BRIEF DESCRIPTION OF THE FIGURES

FIG. 1 depicts a reaction scheme that results in a mixture of regioisomeric ketones via hydrolysis of an aryl alkyne.

FIG. 2 depicts [A] reaction steps and conditions from the chemical literature that failed in coupling an aryl bromide containing an unprotected amino alcohol with a hydrazone; and [B] reaction steps and conditions of the present invention that succeeded in providing the desired final product.

FIG. 3 depicts selected reactions of the invention.

FIG. 4 depicts the oxidation of an alcohol to an aldehyde; and the subsequent formation of a hydrazone from the aldehyde.

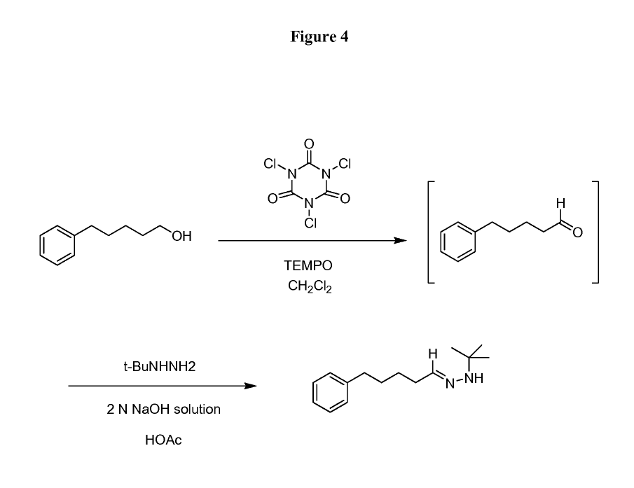

FIG. 5 tabulates selected reaction conditions and results for the reduction of an amino ester to an amino alcohol.

FIG. 6 depicts a metal-catalyzed coupling of a hydrazone and an aryl bromide to form an aryl ketone, and selected steps in the preparation of the hydrazone and aryl bromide.

FIG. 7 depicts an example of a Sonogashira coupling of a terminal alkyne and an aryl bromide.

### DETAILED DESCRIPTION

The present invention is directed in part to methods of making compounds which are agonists or antagonists of the individual receptors of the S1P receptor family.

### DEFINITIONS

In this invention, the following definitions are applicable:

Certain compounds of the invention which have basic substituents may exist as salts with acids (e.g, primary amines). The present invention includes such salts. Examples of such salts include salts which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

Certain compounds of the invention which have acidic substituents may exist as salts with bases. The present invention includes such salts. Examples of such salts include sodium salts, potassium salts, lysine salts and arginine salts. These salts may be prepared by methods known to those skilled in the art.

Certain compounds of the invention and their salts may exist in more than one crystal form and the present invention includes each crystal form and mixtures thereof.

Certain compounds of the invention and their salts may also exist in the form of solvates, for example hydrates, and the present invention includes each solvate and mixtures thereof.

Certain compounds of the invention may contain one or more chiral centers, and exist in different optically active forms. When compounds of the invention contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step may be used to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of the invention contains more than one chiral center, the compound may exist in diastereoisomeric forms. The diastereoisomeric compounds may be separated by methods known to those skilled in the art, for example chromatography or crystallization and the individual enantiomers may be separated as described above. The present invention includes each diastereoisomer of compounds of the invention and mixtures thereof.

Certain compounds of the invention may exist in different tautomeric forms or as different geometric isomers, and the present invention includes each tautomer and/or geometric isomer of compounds of the invention and mixtures thereof.

Certain compounds of the invention may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of the invention and mixtures thereof.

Certain compounds of the invention may exist in zwitterionic form and the present invention includes each zwitterionic form of compounds of the invention and mixtures thereof.

For purposes of this invention, the chemical elements are identified in accordance with the Periodic Table of the Elements, CAS version, Handbook of Chemistry and Physics, 67th Ed., 1986-87, inside cover.

The articles “a” and “an” are used herein to refer to one or to more than one (i.e., to at least one) of the grammatical object of the article. By way of example, “an element” means one element or more than one element.

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” means an alkoxy group, as defined herein, appended to the parent molecular moiety through a carbonyl group, represented by —C(═O)—, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “arylalkoxy” and “heteroalkoxy” as used herein, means an aryl group or heteroaryl group, as defined herein, appended to the parent molecular moiety through an alkoxy group, as defined herein. Representative examples of arylalkoxy include, but are not limited to, 2-chlorophenylmethoxy, 3-trifluoromethylethoxy, and 2,3-methylmethoxy.

The term “arylalkyl” as used herein, means an aryl group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, and n-hexyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” and “arylcarbonyloxy” as used herein, means an alkylcarbonyl or arylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy. Representative examples of arylcarbonyloxy include, but are not limited to phenylcarbonyloxy.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amido” as used herein, means —NHC(═O)—, wherein the amido group is bound to the parent molecular moiety through the nitrogen. Examples of amido include alkylamido such as CH₃C(═O)N(H)— and CH₃CH₂C(═O)N(H)—.

The term “amino” as used herein, refers to radicals of both unsubstituted and substituted amines appended to the parent molecular moiety through a nitrogen atom. The two groups are each independently hydrogen, alkyl, alkylcarbonyl, alkylsulfonyl, arylcarbonyl, or formyl. Representative examples include, but are not limited to methylamino, acetylamino, and acetylmethylamino

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

The term “aryl,” as used herein, means a phenyl group or a naphthyl group. The aryl groups of the present invention can be optionally substituted with one, two, three, four, or five substituents independently selected from the group consisting of alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “arylalkyl” or “aralkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkoxy” or “arylalkyloxy” as used herein, means an arylalkyl group, as defined herein, appended to the parent molecular moiety through an oxygen. The term “heteroarylalkoxy” as used herein, means an heteroarylalkyl group, as defined herein, appended to the parent molecular moiety through an oxygen.

The term “arylalkylthio” as used herein, means an arylalkyl group, as defined herein, appended to the parent molecular moiety through an sulfur. The term “heteroarylalkylthio” as used herein, means an heteroarylalkyl group, as defined herein, appended to the parent molecular moiety through an sulfur.

The term “arylalkenyl” as used herein, means an aryl group, as defined herein, appended to the parent molecular moiety through an alkenyl group. A representative example is phenylethylenyl.

The term “arylalkynyl” as used herein, means an aryl group, as defined herein, appended to the parent molecular moiety through an alkynyl group. A representative example is phenylethynyl.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “arylcarbonylalkyl” as used herein, means an arylcarbonyl group, as defined herein, bound to the parent molecule through an alkyl group, as defined herein.

The term “arylcarbonylalkoxy” as used herein, means an arylcarbonylalkyl group, as defined herein, bound to the parent molecule through an oxygen.

The term “aryloxy” as used herein, means an aryl group, as defined herein, appended to the parent molecular moiety through an oxygen. The term “heteroaryloxy” as used herein, means a heteroaryl group, as defined herein, appended to the parent molecular moiety through an oxygen.

The term “carbonyl” as used herein, means a —C(═O)— group.

The term “carboxy” as used herein, means a —CO₂H group.

The term “cycloalkyl” as used herein, means monocyclic or multicyclic (e.g., bicyclic, tricyclic, etc.) hydrocarbons containing from 3 to 12 carbon atoms that is completely saturated or has one or more unsaturated bonds but does not amount to an aromatic group. Examples of a cycloalkyl group include cyclopropyl, cyclobutyl, cyclopentyl, cyclopentenyl, cyclohexyl and cyclohexenyl.

The term “cycloalkoxy” as used herein, means a cycloalkyl group, as defined herein, appended to the parent molecular moiety through an oxygen.

The term “cyano” as used herein, means a —CN group.

The term “formyl” as used herein, means a —C(═O)H group.

The term “halo” or “halogen” means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocyclyl”, as used herein, include non-aromatic, ring systems, including, but not limited to, monocyclic, bicyclic and tricyclic rings, which can be completely saturated or which can contain one or more units of unsaturation, (for the avoidance of doubt, the degree of unsaturation does not result in an aromatic ring system) and have 3 to 12 atoms including at least one heteroatom, such as nitrogen, oxygen, or sulfur. For purposes of exemplification, which should not be construed as limiting the scope of this invention, the following are examples of heterocyclic rings: azepinyl, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl. The heterocyclyl groups of the invention are optionally substituted with 0, 1, 2, or 3 substituents independently selected from, for example, alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroaryl” as used herein, include aromatic ring systems, including, but not limited to, monocyclic, bicyclic and tricyclic rings, and have 3 to 12 atoms including at least one heteroatom, such as nitrogen, oxygen, or sulfur. For purposes of exemplification, which should not be construed as limiting the scope of this invention: azaindolyl, benzo(b)thienyl, benzimidazolyl, benzofuranyl, benzoxazolyl, benzothiazolyl, benzothiadiazolyl, benzotriazolyl, benzoxadiazolyl, furanyl, imidazolyl, imidazopyridinyl, indolyl, indolinyl, indazolyl, isoindolinyl, isoxazolyl, isothiazolyl, isoquinolinyl, oxadiazolyl, oxazolyl, purinyl, pyranyl, pyrazinyl, pyrazolyl, pyridinyl, pyrimidinyl, pyrrolyl, pyrrolo[2,3-d]pyrimidinyl, pyrazolo[3,4-d]pyrimidinyl, quinolinyl, quinazolinyl, triazolyl, thiazolyl, thiophenyl, tetrahydroindolyl, tetrazolyl, thiadiazolyl, thienyl, thiomorpholinyl, triazolyl or tropanyl. The heteroaryl groups of the invention are optionally substituted with 0, 1, 2, or 3 substituents independently selected from alkenyl, alkoxy, alkoxycarbonyl, alkoxysulfonyl, alkyl, alkylcarbonyl, alkylcarbonyloxy, alkylsulfonyl, alkylthio, alkynyl, amido, amino, carboxy, cyano, formyl, halo, haloalkoxy, haloalkyl, hydroxyl, hydroxyalkyl, mercapto, nitro, silyl and silyloxy.

The term “heteroarylalkyl” or “heteroaralkyl” as used herein, means a heteroaryl, as defined herein, appended to the parent molecular moiety through an alkyl group, as defined herein. Representative examples of heteroarylalkyl include, but are not limited to, pyridin-3-ylmethyl and 2-(thien-2-yl)ethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “mercapto” as used herein, means a —SH group.

The term “nitro” as used herein, means a —NO₂ group.

The term “silyl” as used herein includes hydrocarbyl derivatives of the silyl (H₃Si—) group (i.e., (hydrocarbyl)₃Si—), wherein a hydrocarbyl groups are univalent groups formed by removing a hydrogen atom from a hydrocarbon, e.g., ethyl, phenyl. The hydrocarbyl groups can be combinations of differing groups which can be varied in order to provide a number of silyl groups, such as trimethylsilyl (TMS), tert-butyldiphenylsilyl (TBDPS), tert-butyldimethylsilyl (TBS/TBDMS), triisopropylsilyl (TIPS), and [2-(trimethylsilyl)ethoxy]methyl (SEM).

The term “silyloxy” as used herein means a silyl group, as defined herein, is appended to the parent molecule through an oxygen atom.

The term “sulfonate” as used herein means —S(═O)₂OR, wherein R is hydrogen, alkyl, alkenyl, alkynyl, aryl, aralkyl, heteroaryl or heteroaralkyl. Examples of sulfonates include tosylates and mesylates.

The term “catalytic amount” is recognized in the art and means a substoichiometric amount of reagent relative to a reactant. As used herein, a catalytic amount means, for example, from 0.0001 to 90 mole percent reagent relative to a reactant, or 0.001 to 50 mole percent, or from 0.01 to 10 mole percent, or from 0.1 to 5 mole percent reagent to reactant.

A “polar solvent” means a solvent which has a dielectric constant (c) of 2.9 or greater, such as DMF, THF, ethylene glycol dimethyl ether (DME), DMSO, acetone, acetonitrile, methanol, ethanol, isopropanol, n-propanol, t-butanol or 2-methoxyethyl ether.

### Preparation of Aryl Ketones

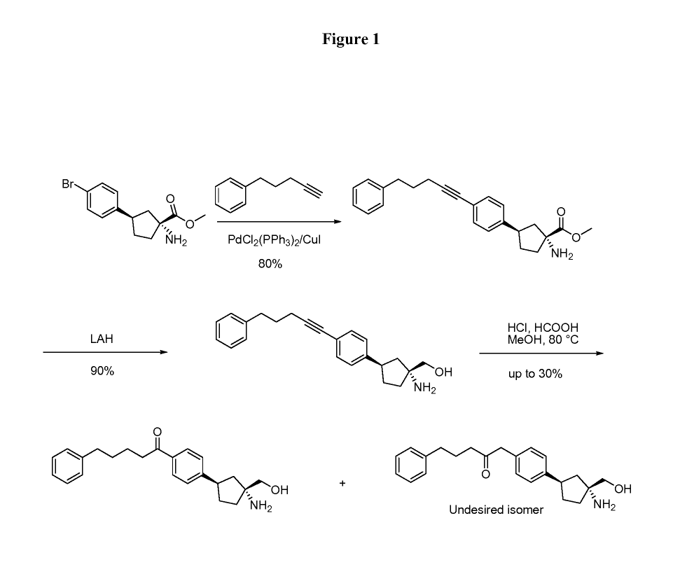

As shown in FIG. 1, aryl ketones may be formed via the hydrolysis of aryl alkynes. However, the hydrolysis of the alkyne often requires the use of harsh chemicals such as sulfuric acid or mercury. In addition, hydrolysis often results in regioisomeric ketones which can be difficult to separate. The some cases the undesired ketone isomer is inseparable from the desired isomer.

Another approach to the preparation of aryl ketones is via a metal-catalyzed coupling of an aryl halide with an acyl anion equivalent. A literature procedure reports the use of Pd₂(dba)₃ (2.5 mol %) and DPEPHOS (5 mol %) as catalyst in the presence of NaOtBu (1.4 equiv.) as base. See Takemiya, A.; and Hartwig, J. F. “Palladium-Catalyzed Synthesis of Aryl Ketones by Coupling of Aryl Bromides with an Acyl Anion Equivalent” J. Am. Chem. Soc. 2006, 128 (46), 14800-14801.

While Takemiya and Hartwig have reported on the Pd-catalyzed cross-coupling reactions of aryl bromides with acyl anion equivalents, it is believed that there have been no reported examples of Pd-catalyzed cross-coupling reaction between an aryl halide containing an unprotected amino alcohol and an acyl anion equivalent. Takemiya and Hartwig show no examples of reactions of aryl bromides containing free amine or alcohol functionalities because free amine and alcohol groups are known to stall palladium-catalyzed reactions. Indeed, the authors in the above reference had to protect the free OH groups in the aryl bromide by TBS protecting group to allow the reaction to proceed. Reactions with aryl bromides containing NH₂ group in either protected or unprotected form were not even attempted. While not intending to be bound by any particular theory, it is hypothesized that in addition to the problem of catalyst poisoning, the free OH and NH₂ groups might be more likely to form C—O and C—N bonds instead of the desired C—C bond in the reaction. While there are thousands of examples of Pd-catalyzed C—O and C—N bond forming reactions, it is believed that there are only two examples of Pd-catalyzed cross-coupling reaction between aryl bromides and acyl anion equivalents, stressing the fact that Pd-catalyzed C—O and C—N bond forming reactions are more facile. Even in the Takemiya and Hartwig reference there is report of competitive C—N bond formation.

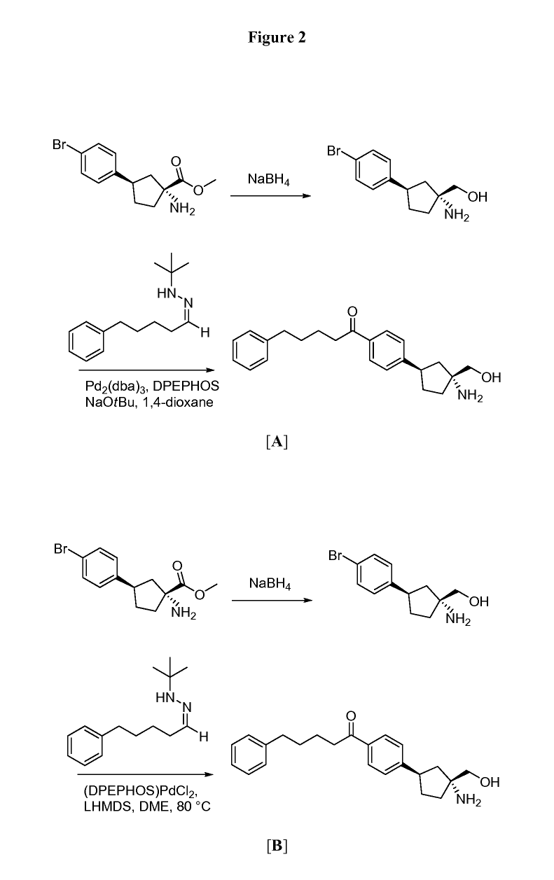

In fact, when the Takemiya and Hartwig reaction conditions are applied to the coupling depicted in FIG. 2A, no appreciable amount of the product was obtained. As noted above, it was hypothesized that the cause of the failure of the reaction might be the unprotected amino alcohol functionality that is known to chelate to Pd and stall the catalytic reaction. Specifically, NaOtBu used as the base in the catalytic reaction was probably deprotonating the amino alcohol functionality and was accelerating the process of catalyst decomposition.

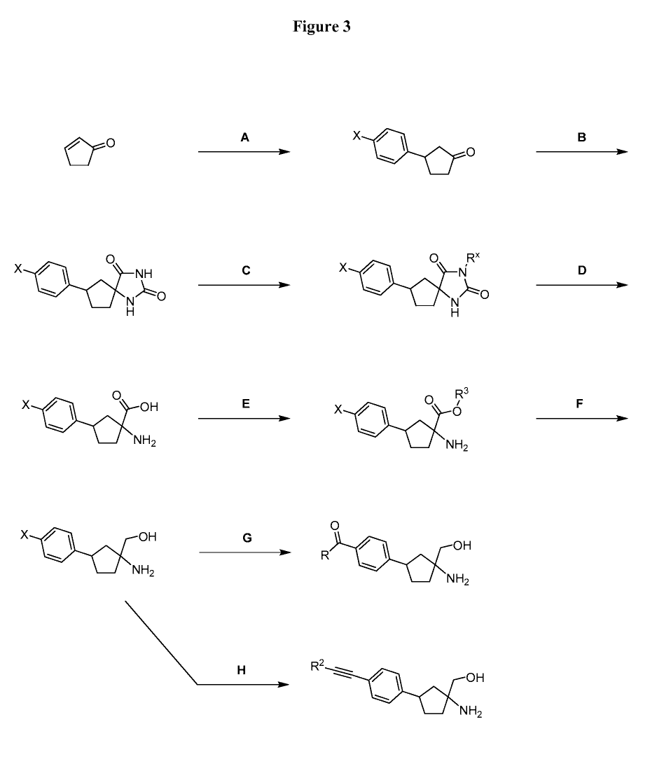

It was realized that performing the reaction under inert conditions might be the key to the success of the reaction. Therefore, the reaction was modified to use LHMDS (lithium hexamethylsilylazide) as the base instead of NaOtBu as the base, as shown in FIG. 3B.

It is believed that prior to the results disclosed herein, the use of LHMDS as base in the Pd-catalyzed cross-coupling reaction of aryl bromides with unprotected amino alcohol and an acyl anion equivalent was unknown. However, the use of LHMDS as a base in a different type of Pd-catalyzed cross-coupling reaction (C—N bond formation) has been reported. See Harris, M. C.; Huang, X.; Buchwald, S. L. “Improved Functional Group Compatibility in the Palladium-Catalyzed Synthesis of Aryl Halides,” Org. Lett. 2002, 4, 2885; and Shen, Q., Ogata, T., and J. F. Hartwig “Highly Reactive, General and Long-Lived Catalysts for Palladium-Catalyzed Amination of Heteroaryl and Aryl Chlorides, Bromides and Iodides: Scope and Structure-Activity Relationships,” J. Am. Chem. Soc. 2008, 130(20), 6586-6596. While it was known that LHMDS deprotonates alcohols and forms a lithium aggregate that allows the cross-coupling reactions to proceed, it has been reported that the cross-coupling reactions fail to proceed, even in the presence of LHMDS as the base, if any of the reactants contain NH₂ functional group.

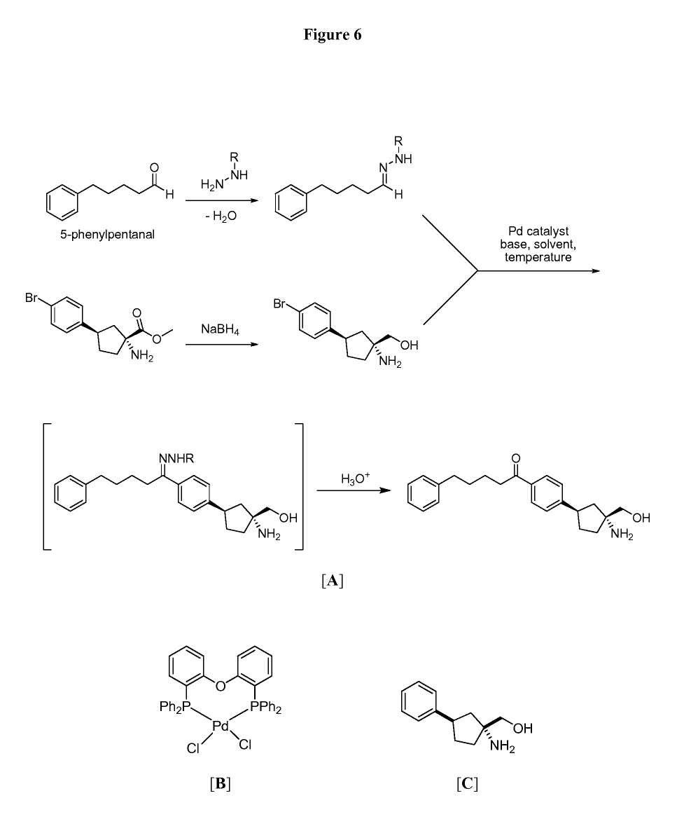

However, while not intending to be bound my any one theory, it was hypothesized that for α-amino alcohol containing compounds the lithium aggregate formed by the deprotonation of OH group might put NH₂ group in a very sterically hindered environment, thereby rendering NH₂ incapable of poisoning the catalyst. Remarkably, this new synthetic approach provided an unprecedented chemistry where a Pd-catalyzed cross-coupling reaction between an aryl bromide containing unprotected amino alcohol and an acyl anion equivalent was achieved. As depicted in FIGS. 3B and 6A, the use of 4 equivalent of LHMDS formed the product in greater than 80% yield with about 2-10% dehalogenated product as a side product (see FIG. 6C). The optimal reaction conditions found to date use (DPEPhos)PdCl₂ (see FIG. 6B) as a catalyst and LHMDS as base in DME as solvent at about 80° C. to form the aryl ketone.

In summary, herein are disclosed reaction conditions that have allowed the metal-catalyzed coupling of aryl bromides containing unprotected amino alcohol functionalities with acyl anion equivalents, such as hydrazones. One of the keys to the success of this reaction was to employ LHMDS as the base instead of NaOtBu.

### Purification Methods

In addition to the formation of the desired aryl ketone, the coupling reaction depicted in FIG. 6 also produced 2-10% of a dehalogenated by-product (see FIG. 6C). This compound is a potentially harmful impurity. A novel work-up procedure was developed to reduce the amount of this impurity. In certain embodiments, the impurity is reduced to less the 0.2 mol % level. Specifically, a work up procedure was developed that involved washing of the HCl salt of the desired compound suspended in CH₂Cl₂ with aqueous K₂CO₃ solutions. The pH of the aqueous layer was carefully maintained between 9-9.5. This method extracted the impurity into the aqueous layer and limited the amount of the impurity in the organic layer to below 0.2 mol % (in some cases with only a 5-6 mol % loss of desired product in the aqueous layer). It was extremely crucial to maintain the pH of the aqueous layer between 9-9.5; higher pH did not lead to extraction of the impurity into the aqueous layer and pH lower than 9 formed an inseparable mixture of aqueous and organic layers. The removal of any unreacted starting material is also expected from the product mixture using this method.

Importantly, highlighting the importance of the purification procedure described above, for certain compounds silica gel column chromatographic techniques are not amenable to scale up and thus are not commercially viable.

### Sonogashira Coupling

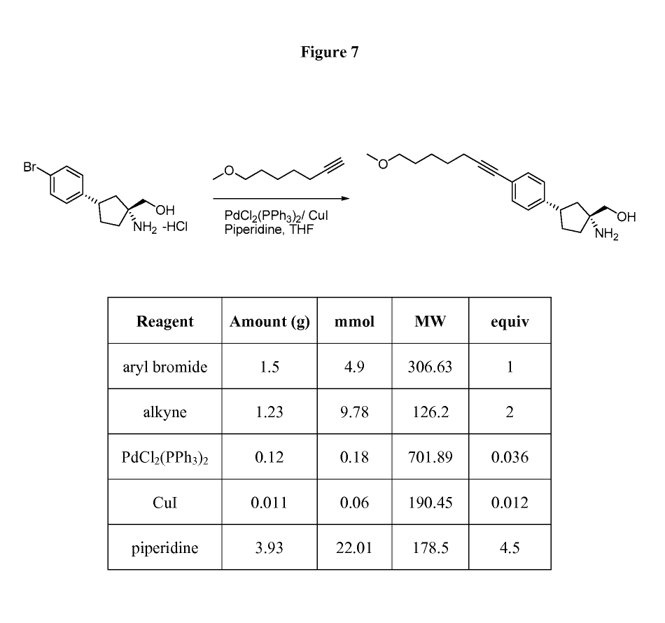

It has also been found that aryl halides containing unprotected amino alcohol functionality can also be coupled to alkynes (Sonogashira couplings), when an excess of LHMDS is used as the base. FIG. 7 depicts one such coupling.

### Various General Considerations

The reactions described herein typically proceed at mild temperatures and pressures to give high yields of the product, such as aryl ketones. Thus, yields of desired products greater than 45%, greater than 75%, and greater than 80%, for example, may be obtained from reactions according to the invention.

The ligands of the present invention and the methods based thereon enable the formation of carbon-carbon bonds—via transition metal catalyzed reactions—under conditions that would not yield appreciable amounts of the observed product(s) using methods known in the art. When a reaction is said to occur under a given set of conditions it means that the rate of the reaction is such the bulk of the starting materials is consumed, or a significant amount of the desired product is produced, for example, within 48 hours, within 24 hours, or within 12 hours. In certain embodiments, the ligands and methods of the present invention catalyze the aforementioned transformations utilizing less than 1 mol % of the catalyst complex relative to the limiting reagent, in certain embodiments less than 0.01 mol % of the catalyst complex relative to the limiting reagent, and in additional embodiments less than 0.0001 mol % of the catalyst complex relative to the limiting reagent.

One aspect of the present invention relates to a transition metal-catalyzed reaction which comprises combining an acyl anion equivalent with a substrate aryl group bearing an activated group X and an α-amino alcohol moiety. The reaction includes at least a catalytic amount of a transition metal catalyst, comprising a ligand, and the combination is maintained under conditions appropriate for the metal catalyst to catalyze the reaction.

Suitable substrate aryl compounds include compounds derived from simple aromatic rings (single or polycyclic) such as benzene, naphthalene, anthracene and phenanthrene; or heteroaromatic rings (single or polycyclic), such as pyrrole, thiophene, thianthrene, furan, pyran, isobenzofuran, chromene, xanthene, phenoxathiin, imidazole, pyrazole, thiazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, perimidine, phenanthroline, phenazine, phenarsazine, phenothiazine, furazan, phenoxazine, pyrrolidine, oxolane, thiolane, oxazole, piperidine, piperazine, morpholine and the like. In certain embodiments, the reactive group, X, is substituted on a five, six or seven membered ring (though it can be part of a larger polycycle).

In certain embodiments, the aryl substrate may be selected from the group consisting of phenyl and phenyl derivatives, heteroaromatic compounds, polycyclic aromatic and heteroaromatic compounds, and functionalized derivatives thereof. Suitable aromatic compounds derived from simple aromatic rings and heteroaromatic rings, include but are not limited to, pyridine, imidazole, quinoline, furan, pyrrole, thiophene, and the like. Suitable aromatic compounds derived from fused ring systems, include but are not limited to naphthalene, anthracene, tetralin, indole and the like.

An activated substituent, X, is characterized as being a good leaving group. In general, the leaving group is a group such as a halide or sulfonate. Suitable activated substituents include, by way of example only, halides such as chloride, bromide and iodide, and sulfonate esters such as triflate, mesylate, nonaflate and tosylate. In certain embodiments, the leaving group is a halide selected from iodine, bromine, and chlorine. In certain embodiments, the leaving group is a sulfonate esters selected from triflate, mesylate, nonaflate and tosylate.

In certain embodiments, the corresponding salt of an amine may be prepared and used in place of the amine.

In certain embodiments, the acyl anion equivalent is a hydrazone. The hydrazone or the like is selected to provide the desired reaction product. The hydrazone or the like may be functionalized. The hydrazone or the like may be selected from a wide variety of structural types, including but not limited to, acyclic, cyclic or heterocyclic compounds, fused ring compounds or phenol derivatives. The aromatic compound and the hydrazone or the like may be included as moieties of a single molecule, whereby the reaction proceeds as an intramolecular reaction.

It is contemplated that the “metal catalyst” of the present invention, as that term is used herein, shall include any catalytic transition metal and/or catalyst precursor as it is introduced into the reaction vessel and which is, if necessary, converted in situ into the active form, as well as the active form of the catalyst which participates in the reaction.

In certain embodiments, the transition metal catalyst complex is provided in the reaction mixture in a catalytic amount. In certain embodiments, that amount is in the range of, for example, 0.0001 to 20 mol %; 0.05 to 5 mol % or 1 to 4 mol %, with respect to the limiting reagent, which may be either the aromatic compound or the acyl anion equivalent, depending upon which reagent is in stoichiometric excess. In the instance where the molecular formula of the catalyst complex includes more than one metal, the amount of the catalyst complex used in the reaction may be adjusted accordingly. By way of example, Pd₂(dba)₃ has two metal centers; and thus the molar amount of Pd₂(dba)₃ used in the reaction may be halved without sacrificing catalytic activity.

As suitable, the catalysts employed in the subject method involve the use of metals which can mediate cross-coupling of the aryl groups ArX and acyl anion equivalents. In general, any transition metal (e.g., having d electrons) may be used to form the catalyst, e.g., a metal selected from one of Groups 3-12 of the periodic table or from the lanthanide series. However, in certain embodiments, the metal will be selected from the group consisting of late transition metals, e.g., from Groups 5-12 or from Groups 7-11. For example, suitable metals include platinum, palladium, iron, nickel, ruthenium and rhodium. The particular form of the metal to be used in the reaction is selected to provide, under the reaction conditions, metal centers which are coordinately unsaturated and not in their highest oxidation state. The metal core of the catalyst should be a zero valent transition metal, such as Pd, with the ability to undergo oxidative addition to Ar—X bond. The zero-valent state, M(O), may be generated in situ, e.g., from M(II).

To further illustrate, suitable transition metal catalysts include soluble or insoluble complexes of palladium. A zero-valent metal center is presumed to participate in the catalytic carbon-carbon bond forming sequence. Thus, the metal center is desirably in the zero-valent state or is capable of being reduced to metal(0). Suitable soluble palladium complexes include, but are not limited to, tris(dibenzylideneacetone) dipalladium [Pd₂(dba)₃], bis(dibenzylideneacetone) palladium [Pd(dba)₂] and palladium acetate.

The coupling can be catalyzed by a palladium catalyst which palladium may be provided in the form of, for illustrative purposes only, Pd/C, PdCl₂, Pd(OAc)₂, (CH₃CN)₂PdCl₂, Pd[P(C₆H₅)₃]₄, and polymer supported Pd(0).

The catalyst will preferably be provided in the reaction mixture as metal-ligand complex comprising a bound supporting ligand, that is, a metal-supporting ligand complex. The ligand effects can be key to favoring, inter alia, the reductive elimination pathway or the like which produces the products, rather than side reactions such as β-hydride elimination. In certain embodiments, the subject reaction employs bidentate ligands such as bisphosphines or aminophosphines. The ligand, if chiral can be provided as a racemic mixture or a purified stereoisomer. In certain instances, a racemic, chelating ligand is used.

The ligand, as described in greater detail below, may be a chelating ligand, such as by way of example only, alkyl and aryl derivatives of phosphines and bisphosphines. The catalyst complex may include additional ligands as required to obtain a stable complex. Moreover, the ligand can be added to the reaction mixture in the form of a metal complex, or added as a separate reagent relative to the addition of the metal.

In certain embodiments of the subject method, the transition metal catalyst includes one or more phosphine ligands, e.g., as a Lewis basic ligand that controls the stability and electron transfer properties of the transition metal catalyst, and/or stabilizes the metal intermediates. Phosphine ligands are commercially available or can be prepared by methods similar to processes known per se. The phosphines can be monodentate phosphine ligands, such as trimethylphosphine, triethylphosphine, tripropylphosphine, triisopropylphosphine, tributylphosphine, tricyclohexylphosphine, trimethyl phosphite, triethyl phosphite, tripropyl phosphite, triisopropyl phosphite, tributyl phosphite and tricyclohexyl phosphite, triphenylphosphine, tri(o-tolyl)phosphine, triisopropylphosphine or tricyclohexylphosphine; or a bidentate phosphine ligand such as 2,2′-bis(diphenylphosphino)-1,1′-binaphthyl (BINAP), 1,2-bis(dimethylphosphino)ethane, 1,2-bis(diethylphosphino)ethane, 1,2-bis(dipropylphosphino)ethane, 1,2-bis(diisopropylphosphino)ethane, 1,2-bis(dibutyl-phosphino)ethane, 1,2-bis(dicyclohexylphosphino)ethane, 1,3-bis(dicyclohexylphosphino)propane, 1,3-bis(diisopropylphosphino)propane, 1,4-bis(diisopropylphosphino)-butane and 2,4-bis(dicyclohexylphosphino)pentane.

Suitable bis(phosphine) compounds include but are in no way limited to (±)-2,2′-bis(diphenylphosphino)-1,1′-binaphthyl (and separate enantiomers), (±)-2,2′-bis(di-p-tolylphosphino)-1,1′-binaphthyl (and separate enantiomers), 1-1′-bis(diphenylphosphino)-ferrocene (dppf), 1,3-bis(diphenylphosphino)propane (dppp), 1,2-bis(diphenylphosphino)-benzene, 2,2′-bis(diphenylphosphino)diphenyl ether, 9,9-dimethyl-4,5-bis(diphenylphosphino)-xanthene (xantphos), and 1,2-bis(diphenylphosphino)ethane (dppe). Hybrid chelating ligands such as (±)-N,N-dimethyl-1-[2-(diphenylphosphino)ferrocenyl]ethylamine (and separate enantiomers), and (±)-(R)-1-[(S)-2-(diphenylphosphino)-ferrocenyl]ethyl methyl ether (and separate enantiomers) are also within the scope of the invention. In certain embodiments the phosphine ligand is bis(diphenylphosphinophenyl)ether or a substituted form thereof.

In general, a variety of bases may be used in practice of certain aspects of the present invention. The base may be sterically hindered to discourage metal coordination of the base in those circumstances where such coordination is possible. In certain embodiments, the base is a bis(trialkylsilyl)amide (e.g., KN(SiMe₃)₂, NaN(SiMe₃)₂, and LiN(SiMe₃)₂).

In certain embodiments, base is used in at least a two fold excess. For the preparation of aryl ketones, the present invention has demonstrated that there is a need for large excesses of base in order to obtain good yields of the desired products. In certain embodiments, three or four equivalents of base are needed.

As is clear to one of skill in the art, the products which may be produced by the reactions of this invention can undergo further reaction(s) to afford desired derivatives thereof. Such permissible derivatization reactions can be carried out in accordance with conventional procedures known in the art. For example, potential derivatization reactions include esterification, oxidation of alcohols to aldehydes and acids, N-alkylation of amides, nitrile reduction, acylation of alcohols by esters, acylation of amines and the like.

The reactions of the present invention may be performed under a wide range of conditions, though it will be understood that the solvents and temperature ranges recited herein are not limitative and only correspond to an exemplary mode of the process of the invention.

In general, it will be desirable that reactions are run using mild conditions which will not adversely affect the reactants, the catalyst, or the product. For example, the reaction temperature influences the speed of the reaction, as well as the stability of the reactants and catalyst. The reactions will usually be run at temperatures in the range of about 25° C. to about 300° C., or in the range about 25° C. to about 150° C.

In general, the subject reactions are carried out in a liquid reaction medium. The reactions may be run without addition of solvent. Alternatively, the reactions may be run in an inert solvent, preferably one in which the reaction ingredients, including the catalyst, are substantially soluble. Suitable solvents include ethers, such as diethyl ether, 1,2-dimethoxyethane, diglyme, t-butyl methyl ether, tetrahydrofuran, water and the like; halogenated solvents, such as chloroform, dichloromethane, dichloroethane, chlorobenzene, and the like; aliphatic or aromatic hydrocarbon solvents such as benzene, xylene, toluene, hexane, pentane and the like; esters and ketones, such as ethyl acetate, acetone, and 2-butanone; polar aprotic solvents such as acetonitrile, dimethylsulfoxide, dimethylformamide and the like; or combinations of two or more solvents.

The invention also contemplates reaction in a biphasic mixture of solvents, in an emulsion or suspension, or reaction in a lipid vesicle or bilayer. In certain embodiments, the reaction is performed with a reactant or ligand anchored to a solid support.

In certain embodiments the reactions are performed under an inert atmosphere of a gas, such as nitrogen or argon.

In certain embodiments the reactions are performed under microwave irradiation. The term “microwave” refers to that portion of the electromagnetic spectrum between about 300 and 300,000 megahertz (MHz) with wavelengths of between about one millimeter (1 mm) and one meter (1 m). These are, of course, arbitrary boundaries, but help quantify microwaves as falling below the frequencies of infrared radiation but above those referred to as radio frequencies. Similarly, given the well-established inverse relationship between frequency and wavelength, microwaves have longer wavelengths than infrared radiation, but shorter than radio frequency wavelengths. Microwave-assisted chemistry techniques are generally well established in the academic and commercial arenas. Microwaves have some significant advantages in heating certain substances. In particular, when microwaves interact with substances with which they can couple, most typically polar molecules or ionic species, the microwaves can immediately create a large amount of kinetic energy in such species which provides sufficient energy to initiate or accelerate various chemical reactions. Microwaves also have an advantage over conduction heating in that the surroundings do not need to be heated because the microwaves can react instantaneously with the desired species.

The reaction processes of the present invention can be conducted in continuous, semi-continuous or batch fashion and may involve a liquid recycle operation as desired. The processes of this invention are preferably conducted in batch fashion. Likewise, the manner or order of addition of the reaction ingredients, catalyst and solvent are also not generally critical to the success of the reaction, and may be accomplished in any conventional fashion. In a order of events that, in some cases, can lead to an enhancement of the reaction rate, the base, e.g., PhONa, is the last ingredient to be added to the reaction mixture.

The reaction can be conducted in a single reaction zone or in a plurality of reaction zones, in series or in parallel or it may be conducted batchwise or continuously in an elongated tubular zone or series of such zones. The materials of construction employed should be inert to the starting materials during the reaction and the fabrication of the equipment should be able to withstand the reaction temperatures and pressures. Means to introduce and/or adjust the quantity of starting materials or ingredients introduced batchwise or continuously into the reaction zone during the course of the reaction can be conveniently utilized in the processes especially to maintain the desired molar ratio of the starting materials. The reaction steps may be affected by the incremental addition of one of the starting materials to the other. Also, the reaction steps can be combined by the joint addition of the starting materials to the metal catalyst. When complete conversion is not desired or not obtainable, the starting materials can be separated from the product and then recycled back into the reaction zone.

The processes may be conducted in either glass lined, stainless steel or similar type reaction equipment. The reaction zone may be fitted with one or more internal and/or external heat exchanger(s) in order to control undue temperature fluctuations, or to prevent any possible “runaway” reaction temperatures.

Furthermore, one or more of the reactants can be immobilized or incorporated into a polymer or other insoluble matrix.

### EXEMPLIFICATION

The invention now being generally described, it will be more readily understood by reference to the following examples, which are included merely for purposes of illustration of certain aspects and embodiments of the present invention, and are not intended to limit the invention.

### Abbreviations

acac Acetylacetonate

### ACN Acetonitrile

BBr₃ Borane tribromide

### C₂H₄ Ethylene

CuI Copper(I) iodide
DBAD Di-tert-butyl azodicarboxylate

### DCM Dichloromethane

de diastereomeric excess
DPEPhos bis(diphenylphosphinophenyl)ether

### DIEA N,N-Diisopropylethylamine

### DMA N,N-Dimethylacetamide

### DME 1,2-Dimethoxyethane

### DMF N,N-Dimethylformamide

DMSO Dimethyl sulfoxide
dppf 1,1′-Bis(diphenylphosphino)ferrocene
ee enantiomeric excess

### Et₃N Triethylamine

Et₂O Diethyl ether
EtOAc Ethyl acetate

### h Hour(s)

### H₂ Hydrogen gas

HCl Hydrochloric acid
HOAc Acetic acid

### HPLC High Performance Liquid Chromatography

K₂CO₃ Potassium carbonate
LAH Lithium tetrahydroaluminate
LDA Lithium diisopropylamide
LiHMDS Lithium hexamethyldisilazide
LiOH Lithium hydroxide

### MeOH Methanol

MgSO₄ Magnesium sulfate
NaHCO₃ Sodium bicarbonate
NaOH Sodium hydroxide
Na₂SO₄ Sodium sulfate
NBD Bicyclo[2.2.1]hepta-2,5-diene
Pd(PPh₃)₂Cl₂ Bis(triphenylphosphine)palladium(II) chloride

### PPh₃ Triphenylphosphine

PS-PPh₃ Polymer-supported triphenylphosphine

### Rh Rhodium

### RP Reverse Phase

Rₜ Retention time
RT Room temperature
(R)-BINAP (R)-(−)-2,2′-Bis(diphenylphosphino)-1,1′-binaphthalene
(S)-BINAP (S)-(+2,2′-Bis(diphenylphosphino)-1,1′-binaphthalene

### THF Tetrahydrofuran

TLC Thin layer chromatography

### Analytical Methods

Analytical data is defined either within the general procedures or in the tables of examples. Unless otherwise stated, all ¹H or ¹³C NMR data were collected on a Varian Mercury Plus 400 MHz or a Bruker DRX 400 MHz instrument; chemical shifts are quoted in parts per million (ppm). High-pressure liquid chromatography (HPLC) analytical data are either detailed within the experimental or referenced to the table of HPLC conditions, using the lower case method letter, in Table 1.

TABLE 1
List of HPLC Methods

| | HPLC Conditions |
|---|---|
| | Unless indicated otherwise mobile phase A was 10 mM ammonium acetate, |
| Method | mobile phase B was HPLC grade acetonitrile. |
| a | 5-95% B over 3.7 min with a hold at 95% B for 1 min (1.3 mL/min flow |
| | rate). 4.6 × 50 mm Waters Zorbaz XDB C18 column (5 μm particles). |
| | Detection methods are diode array (DAD) and evaporative light scattering |
| | (ELSD) detection as well as pos/neg electrospray ionization. |
| b | 5-60% B over 1.5 min then 60-95% B to 2.5 min with a hold at 95% B for |
| | 1.2 min (1.3 mL/min flow rate). 4.6 × 30 mm Vydac Genesis C8 column (4 |
| | μm particles). Detection methods are diode array (DAD) and evaporative |
| | light scattering (ELSD) detection as well as pos/neg electrospray ionization. |
| c | 5-60% B over 1.5 min then 60-95% B over 2.5 min with a hold at 95% B for |
| | 1.2 min (1.3 mL/min flow rate). 4.6 × 50 mm Zorbax XDB C8 column (5 |
| | μm particles). Detection methods are diode array (DAD) and evaporative |
| | light scattering (ELSD) detection as well as pos/neg electrospray ionization. |
| d | 30% to 95% B over 2.0 min; 95% B for 1.5 min at 1.0 mL/min; UV λ = 210- |
| | 360 nm; Genesis C8, 4 μm, 30 4.6 mm column; ESI +ve/−ve) |
| e | 10% to 40% B over 4.0 min; 40% to 95% B over 2.0 min; 95% B for 1.0 min |
| | at 1.0 mL/min; UV λ = 210-360 nm; Genesis C8, 4 μm, 30 × 4.6 mm |
| | column; ESI +ve/−ve) |
| f | 5% to 95% B over 2.0 min; 95% B for 1.5 min at 1.4 mL/min; UV λ = 210- |
| | 360 nm; Genesis C8, 4 μm, 30 × 4.6 mm column; ESI +ve/−ve) |
| h | 30% to 95% B over 2.0 min; 95% B for 3.5 min at 1.0 mL/min; UV λ = 190- |
| | 400 nm; 4.6 × 30 mm Vydac Genesis C8 column (4 μm particles); Detection |
| | methods are diode array (DAD) and evaporative light scattering (ELSD) |
| | detection as well as pos/neg electrospray ionization. |
| i | 5% to 35% B over 4.0 min; 35%-95% B over 2 min; 95% B for 1.0 min at |
| | 1.0 mL/min; UV λ = 190-400 nm; Genesis C8, 4 μm, 30 × 4.6 mm column; |
| | ESI +ve/−ve) |

### General Synthetic Schemes/Procedures

General synthetic schemes that were utilized to construct the majority of compounds disclosed in this application are shown in the Figures.

The following describes general synthetic procedures and examples of compounds that were synthesized following the general procedures. Unless noted otherwise, none of the specific conditions and reagents noted in the following are to be construed as limiting the scope of the instant invention and are provided for illustrative purposes only. All of the general procedures have been successfully performed and exemplifications of each general procedure is also provided.

### General Procedure A

### Michael Addition to an Alpha-Beta Unsaturated Ketone

A solution of substituted arylboronic acid (1-3 equilvalents, preferably 1.5 equivalents) and a rhodium catalyst (such as Rh(NBD)(S-BINAP)BF₄, hydroxyl[(S)-BINAP]rodium(I) dimer, Rh(acac)(C₂H₄)₂/(R)-BINAP, or acetylacetonatobis(ethylene)rhodium(I) with (R)- or (S)-BINAP, preferably Rh(NBD)(S-BINAP)BF₄ for (S)-product, Rh(acac)(C₂H₄)₂/(R)-BINAP for (R)-product) (1-5 mol %, preferably 1.25 mol %) in an organic solvent (such as tetrahydrofuran, or dioxane, preferably dioxane) and water is degassed with nitrogen. A cycloalkanone is added to the mixture. The reaction is stirred at about 20-100° C. (such as at about 35° C.) for a period of 1-24 h (such as for 16 h) under inert atmosphere with or without the addition of an organic base (preferably triethylamine). The reaction mixture is concentrated under reduced pressure and the crude product is purified via flash chromatography.

### Exemplification of General Procedure A

### Preparation of (S)-3-(4-Bromo-phenyl)-cyclopentanone

Rh(NBD)(S-BINAP)BF₄ (22 mg) and S-BINAP (40 mg) are mixed together in degassed 1,4-dioxane (3 mL). The mixture is stirred for about 2 h at RT to give an orange slurry. In a separate flask, 4-bromophenylboronic acid (1 g, 1.5 equiv) is dissolved in dioxane (5.6 mL) and water (1.4 mL) at RT, and then transferred into the flask containing the catalyst. The resulting suspension is degassed with nitrogen and 2-cyclopenten-1-one (0.273 g, 1 equiv) and triethylamine (0.336 g, 1 equiv) are added. The red-orange clear solution is stirred overnight at RT. The reaction is separated between ethyl acetate and water, and the organic layer is washed once with 5% NaCl(aq), then concentrated. The crude product is further purified on silica gel column using 20% ethyl acetate in heptanes.

Alternatively, a 3 L three-necked round bottom flask equipped with temperature probe and nitrogen bubbler was charged with 4-bromophenylboronic acid (100 g, 498 mmol) and hydroxyl[(S)-BINAP]rhodium(I) dimer (6.20 g, 4.17 mmol) in dioxane (1667 mL) and water (167 mL) at RT. The resulting suspension was degassed with nitrogen and 2-cyclopenten-1-one (27.8 mL, 332 mmol) was added in one portion. The mixture was further degassed for 5 minutes and heated at about 35° C. for about 16 h. The reaction mixture was cooled to RT and concentrated. The brown residue was treated with EtOAc (500 mL) and filtered. The filtrate was washed with a saturated solution of NaHCO₃ (500 mL) and brine (500 mL), dried over MgSO₄, filtered, and concentrated to afford a dark brown solid. The crude reaction product was product was purified by silica gel chromatography (1:9 EtOAc:heptane as eluant). Fractions containing product were combined and concentrated to afford (S)-3-(4-bromo-phenyl)-cyclopentanone (70.4 g, 89%, 95% ee as determined by chiral HPLC) as an ivory solid.

LCMS (Table 1, Method a) Rₜ=2.81 min; no characteristic mass detected; ¹H NMR (400 MHz, DMSO-d₆) δ 7.47 (d, 2H), 7.27 (d, 2H), 3.35 (m, 1H), 2.55 (m, 1H), 2.25 (m, 4H), 1.85 (m, 1H)

Alternatively, the boronate can be formed in situ and used in the rhodium catalyzed addition to an enone as follows. A 250 mL round-bottomed flask equipped with a rubber septum and nitrogen inlet needle is charged with 1-bromo-4-octylbenzene (5.77 g, 21.43 mmol) in Et₂O (10.7 mL) at RT. The resulting solution is cooled to about 0° C. After about 5 min BuLi (8.21 mL, 21.43 mmol) solution is added dropwise via syringe over about 20 min. The reaction mixture was allowed to stir at about 0° C. for about 30 min. The resulting solution is then cooled to about −78° C. After about 10 min trimethyl borate (2.395 mL, 21.43 mmol) is added dropwise via syringe over about 5 min. The reaction mixture is allowed to stir at about −78° C. for about 30 min. The reaction mixture is treated with 20 mL of saturated NH₄Cl and 50 mL of toluene. The aqueous phase is separated and extracted with two 50-mL portions of toluene. The organic phases are combined and concentrated. The residue is further diluted with toluene and concentrated to remove water and then dried in vacuo. The resulting white pasty solid is used directly in the next transformation. The crude borate is transferred to a 200 mL round-bottomed flask equipped with a reflux condenser outfitted with a nitrogen inlet adapter while acetylacetonatobis(ethylene)rhodium(I) (0.166 g, 0.643 mmol) and (R)-BINAP enantiomer (0.480 g, 0.772 mmol) are added in one portion each. The flask is evacuated and filled with nitrogen (three cycles to remove oxygen). To the solids is added dioxane (40 mL), cyclopent-2-enone (1.796 mL, 21.43 mmol), and water (4 mL) each dropwise via syringe. The resulting suspension is heated at about 100° C. for about 16 h.

The resulting orange/brown solution is allowed to cool to RT. The orange/brown solution is concentrated and the brown residue is taken up in ether and washed with 1N HCl solution. A tan emulsion forms. The emulsified mixture is separated and extracted with EtOAc. The aqueous phases are also extracted with EtOAc. The combined organic phases are washed with 10% NaOH and Brine, then concentrated to afford a brown oil. The crude sample is purified via chromatography on silica gel to afforded 1258 mg of colorless oil.

### General Procedure B

Formation of a Hydantoin from a Ketone

To a mixture of ammonium carbonate (1-10 equivalents, preferably 4.5 equivalents) and a cyanide salt (such as potassium cyanide, or sodium cyanide) (1-3 equivalents, such as 1.1 equivalents) in water is added a ketone (1 equivalent). The reaction mixture is heated to reflux for a period of 2-40 h (such as 16 h). The reaction mixture is cooled to RT and the solid is collected by filtration, and washed with water to give the crude product which can be purified by trituration with ether.

### Exemplification of General Procedure B

### Preparation of (S)-7-(4-bromo-phenyl)-1,3-diaza-spiro[4.4]nonane-2,4-dione

To a round bottom flask charged with ammonium carbonate (268 g, 2.79 mol) and potassium cyanide (44.4 g, 0.681 mol) was added water (1500 mL, 82 mol). The mixture was heated at about 80° C. and a solution of (S)-3-(4-bromo-phenyl)-cyclopentanone (148.09 g, 0.62 mol) in ethanol (1500 mL, 25 mol) was added. The reaction mixture was heated to reflux overnight. The reaction mixture was cooled to RT. The crude reaction mixture was filtered and washed with water. The solid was triturated with ether (1.5 L), filtered, washed with ether and dried under vacuum to yield (S)-7-(4-bromo-phenyl)-1,3-diaza-spiro[4.4]nonane-2,4-dione (181.29 g, 95%) as a 1:1 mixture of diastereomers.

LCMS (Table 1, Method a) Rₜ=2.24 min; m/z: 307 (M−H)⁻; ¹H NMR (400 MHz, DMSO-d₆) δ 10.61 (s, 1H), 8.29 (s, 1H), 8.24 (s, 1H), 7.49 (d, 2H), 7.27 (d, 1H), 7.24 (d, 1H), 3.14-3.35 (m, 1H), 2.45 (dd, 0.5H), 1.68-2.27 (m, 5.5H)

### General Procedure C

### Formation of an N-Alkylated Hydantoin

To a flask containing the hydantoin (1 equivalent) is added a base (such as potassium carbonate, or sodium carbonate) (1-3 equivalents, such as 1.5 equivalents) and an organic solvent such as DMF or DMA. The mixture is stirred at RT for a period of 10-30 minutes (preferably about 15 minutes), then methyl iodide (1-2 equivalents, such as 1.1 equivalents) is added. The reaction is stirred at RT for a period of 24-72 h (such as about 48 h). The reaction mixture is concentrated, cooled in an ice-water bath, and water is added. The precipitate is collected by filtration to give the crude product. The two stereoisomers can be separated by crystallization.

### Exemplification of General Procedure C

### Preparation of (5R,7S)-7-(4-bromo-phenyl)-3-methyl-1,3-diaza-spiro[4.4]nonane-2,4-dione

To the flask containing (S)-7-(4-bromo-phenyl)-1,3-diaza-spiro[4.4]nonane-2,4-dione (1:1 mixture of diastereomers, 180.3 g, 0.583 mol) was added potassium carbonate (120.9 g, 0.875 mol) followed by DMF (1 L). After stirring for about 15 minutes at RT, methyl iodide (39.9 mL, 0.642 mol) was added in one portion. The reaction was stirred at RT over two days. The reaction mixture was partially concentrated in vacuo at about 25° C., removing approximately 400 mL of DMF and excess methyl iodide. The crude mixture was cooled in an ice water bath and water (2 L) was added. After stirring for about 1 h the resulting white precipitate was filtered and rinsed with water (1 L). The filter cake was dried on house vacuum overnight to give 220 g crude (S)-7-(4-bromo-phenyl)-3-methyl-1,3-diaza-spiro[4.4]nonane-2,4-dione as a mixture of diastereomers.

The two diastereomers were separated by crystallization as follows. The material was separated into 2 batches of 110 g each. The crude material (110 g) was suspended in ACN (2.5 L), heated to about 70° C. until near complete dissolution occurred. The material was filtered rapidly at about 70° C. and rinsed with about 70° C. ACN (2×500 mL). The combined filtrates (3.5 L total vol.) were reheated to about 65° C. with stirring. After a clear solution was obtained the mixture was allowed to cool slowly to about 50° C. at which point material began to drop out of solution. The solution was allowed to slowly cool to about 30° C. with stirring (100 rpm). After aging for about 2 h the solution was filtered and the solid was dried at about 65° C. under house vacuum for three h to give (5R,7S)-7-(4-bromo-phenyl)-3-methyl-1,3-diaza-spiro[4.4]nonane-2,4-dione (22.2 g, 12%). (Note: During an attempt to recrystallize from acetonitrile, a mixture of the N-methyl hydantoins enriched in the (S,S)-diastereomer (2:1 (S,S):(R,S)), a small amount of the (5S,7S)-7-(4-bromo-phenyl)-3-methyl-1,3-diaza-spiro[4.4]nonane-2,4-dione (40 mg) in pure form was isolated.)

LCMS (Table 1, Method a) Rₜ=2.50 min; m/z: 321 (M−H)⁻; ¹H NMR (400 MHz, DMSO-d₆) δ 8.56 (s, 1H), 7.50 (d, 2H, J=8.42 Hz), 7.27 (d, 2H, J=8.53 Hz), 3.16-3.31 (m, 1H), 2.84 (s, 3H), 2.46 (dd, 1H, J=13.62, 8.40 Hz,), 2.02-2.18 (m, 2H), 1.72-1.95 (m, 3H)

### Preparation of (5R,7S)-3-allyl-7-(4-bromo-phenyl)-1,3-diaza-spiro[4.4]nonane-2,4-dione

A mixture of isomeric hydantoins (9.27 g, 30 mmol, dried to KF<0.4%), potassium carbonate powder (4.6 g, 33 mmol), allyl bromide (3.8 g, 31.5 mmol) and DMF (45 mL) was agitated overnight at RT. Upon completion (HPLC) the reaction was diluted with water (45 mL), and the slurry was transferred into water (180 mL). The product was collected by filtration, washed with water, 1:1 methanol-water and dried at 50° C. under vacuum to 10.8 g, 103% of white solid.

Allylhydantoin (1:1 mixture of isomers, 10.5 g) was dissolved in dioxane (63 mL) (heating might be required). The desired isomer was precipitated by water addition (40 mL) and mixing the contents for about 4 h at RT. The product was collected by filtration and dried at about 55° C., in vacuo to 2.8 g (10:1 isomers ratio by HPLC) of white solid.

TLC indicated reasonable separation of isomers in the liquors with 65:35 heptanes/EA.

### General Procedure D

### Hydrolysis of a Hydantoin to the Corresponding Amino Acid

To a suspension of N-alkylated hydantoin (1 equivalent) in a mixture of water and organic solvent (such as water/dioxane or water/DMSO) is added an inorganic base (such as lithium hydroxide, or sodium hydroxide) (5-15 equivalents, such as about 8-10 equivalents). The mixture is heated to reflux for a period of 16-48 h (such as about 24 h). After cooling to RT, the reaction mixture is diluted, acidified, and filtered. The filter cake was washed with a suitable solvent (such as water, ethyl acetate or methanol), if necessary, slurried in toluene to remove excess water, and dried under vacuum.

### Exemplification of General Procedure D

### Preparation of (1R,3S)-1-amino-3-(4-bromo-phenyl)-cyclopentanecarboxylic acid

To a slurry of (5R,7S)-7-(4-bromo-phenyl)-3-methyl-1,3-diaza-spiro[4.4]nonane-2,4-dione (79 g, 0.24 mol) in water (1 L) was added 2 M aqueous NaOH (1 L, 2 mol) and dioxane (200 mL). The resulting mixture was heated to reflux for about 24 h. The reaction mixture was cooled to RT, diluted with water (2 L) and acidified with concentrated HCl until a precipitate began to form (about pH 7). Acetic acid (about 20 mL) was added, producing a thick precipitate. The white precipitate was collected and washed with water (2×1 L) and EtOAc (1 L). The filter cake was suspended in toluene (1 L) and concentrated in vacuo at about 45° C. This process was repeated once more. The white precipitate was dried to a constant weight under vacuum to give (1R,3S)-1-amino-3-(4-bromo-phenyl)-cyclopentanecarboxylic acid (65 g, 95%).

LCMS (Table 1, Method a) Rₜ=1.56 min; m/z: 284/286 (M+H)⁺; ¹H NMR (400 MHz, DMSO-d₆) δ 7.55 (d, 2H), 7.3 (d, 2H), 3.3 (m, 1H), 2.65 (m, 1H), 2.3 (m, 1H), 2.1-2.2 (m, 2H), 2.0-2.1 (m, 1H), 1.85 (t, 1H)

Alternatively, the allylhydantoin from above (2.65 g, 7.6 mmol) was dissolved in DMSO (15 mL) and combined with lithium hydroxide solution prepared from LiOH (3.63 g, 150 mmol) and water 50 (mL). The resulting mixture was heated to reflux (105° C.) for about 17 h. Upon completion (HPLC) the reaction mixture was cooled to RT and pH was adjusted to about 7 with concentrated HCl, and then to about 5 with acetic acid (caution foaming!). The product was collected by filtration, washed with water, 1:1 methanol-water and dried to 2.6 g (108%) of grayish solid suitable for the ester formation step.

### General Procedure E

Formation of an Ester from an Acid

An acid (1 equivalent) suspended in large excess of methanol is cooled in an ice/water bath and thionyl chloride (5-20 equivalents, such as 8-12 equivalents) is added dropwise. The resulting mixture is heated to reflux for a period of 2-48 h (such as 24-36 h). The reaction mixture is cooled to RT, filtered and concentrated to dryness. The residue is triturated with a suitable solvent (such as EtOAc or ether) and dried under vacuum to give the desired product.

### Exemplification of General Procedure E

### Preparation of (1R,3S)-1-amino-3-(4-bromo-phenyl)-cyclopentanecarboxylic acid methyl ester; hydrochloride

The (1R,3S)-1-amino-3-(4-bromo-phenyl)-cyclopentanecarboxylic acid (79 g, 0.28 mol) suspended in MeOH (1.8 L) was cooled in an ice/water bath and thionyl chloride (178 mL, 2.44 mol) was added dropwise. Following the addition the reaction was heated to reflux, resulting in a nearly homogeneous solution. After 2 days the reaction mixture was cooled to RT, filtered, and rinsed with MeOH (2×200 mL). The filtrate was concentrated in vacuo to provide a white solid. The white solid was triturated with EtOAc (1 L), collected by filtration, rinsed with EtOAc (2×500 mL), and dried under vacuum to give the (1R,3S)-1-amino-3-(4-bromo-phenyl)-cyclopentanecarboxylic acid methyl ester; hydrochloride as a white solid (79 g, 96%).

LCMS (Table 1, Method a) Rₜ=1.80 min (ELSD); m/z: 198 (M+H)⁺; ¹H NMR (400 MHz, DMSO-d₆) δ 7.55 (d, 2H), 7.35 (d, 2H), 3.82 (s, 3H), 3.3 (m, 1H), 2.65 (m, 1H), 2.3 (m, 1H), 2.1-2.2 (m, 3H), 1.95-2.05 (t, 1H)

17.72 g (62.3 mmol) of crude racemic (S)-1-amino-3-(4-bromophenyl)cyclopentane-carboxylic acid is slurried in MeOH (267 ml), then cooled to about 5° C. Thionyl chloride (27.5 mL, 374 mmol) is added dropwise. Following the addition the reaction mixture is heated to reflux. After about 3-4 h the reaction mixture is cooled to RT and filtered through a Celite® pad. The filtrate is concentrated in vacuo to near dryness and slurried with 100 mL EtOAc followed by removal of ethyl acetate in vacuo. The crude product is slurried in 3% H₂O/EtOAc for about 20 min and filtered to provide 15.88 g white solid. The wetcake is then taken in 270 ml 4% H₂O/DME (Kf=5-6%) and heated to about 50° C. for about 3 h then stirred overnight at RT. The enriched stereoisomer is filtered to provide 7.8 g (37%) (3S,1R) Amino Ester with >98% de. Chiral HPLC showed EtOAc Liquor and DME Liquor with 1:8 ratio and 1:6 ratio (3S,1R):(3S,1S) respectively.

### General Procedure F

### Reducing an α-Amino Ester to an α-Amino Alcohol

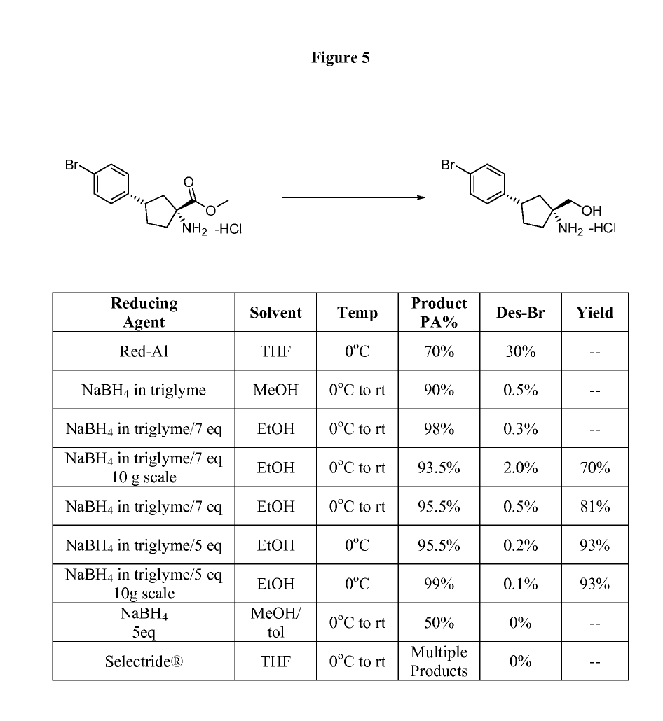

As shown in FIG. 5, several different reducing agents (such as sodium borohydride) were investigated to reduce an amino ester to an amino alcohol while not reducing the halo-aryl bond (for example, to prepare ((1R,3S)-1-amino-3-(4-bromophenyl)cyclopentyl)methanol hydrochloride).

### General Procedure G

### Procedure for Preparing a Hydrazone

An alcohol is dissolved in an organic solvent (such as dichloromethane) and TCAA, and TEMPO is added slowly. The reaction is allowed to stir at RT until the aldehyde is formed (such as for about 15 minutes). The crude reaction mixture is dried and concentrated. A hydrazine hydrochloride is added to 2 N NaOH and stirred until it is dissolved. The crude aldehyde is then added and the reaction mixture is stirred (such as for about 15 minutes). Acetic acid is then added and the reaction mixture is stirred for 12-24 h. The resulting reaction mixture is dried and concentrated.

### Exemplification of General Procedure G

### Preparation of 1-tert-butyl-2-(5-phenylpentylidene)hydrazine

5-Phenylpentanol was dissolved in dichloromethane and TCCA was added. The reaction was cooled and TEMPO was added slowly. After about 15 minutes, the reaction was complete. The workup consists of washing with concentrated sodium carbonate solution, then 1N HCl, and finally brine. The organic is dried and concentrated to an orange oil which is used in the next step as is (about 95% yield). In all cases, reaction preceded as expected. The aldehyde is not stable neat, but is stable as a dichloromethane solution.

The dichloromethane solution from above is concentrated. The t-butyl hydrazine is added to 2 N NaOH and stirred until fully dissolved. The neat aldehyde from the previous step is added and stirred for about 10 minutes. Finally, acetic acid is added and the reaction is stirred overnight. The aqueous is extracted with diethyl ether twice. The organic is washed twice with brine, dried, and concentrated to a white solid. The reaction was completed overnight, but not at 3 h as suggested in the literature.

### General Procedure H

### Pd-Catalyzed Coupling in the Presence of an Excess of a Bis(trialkylsilyl)amide

All the glassware is oven dried prior to use. The solvent to be used is purged with argon for at least 1 h prior to use. A flask equipped with a magnetic stirrer and thermocouple and is charged with a catalyst. The catalyst flask is purged with argon. A separate flask containing a magnetic stir bar is taken inside an inert atmosphere glove box and is charged with a bis(trialkylsilyl)amide. The base flask is brought outside the glove box and an aryl halide is added to the flask followed by the addition of the solvent. The reaction mixture was stirred at RT for about 30 min while being purged with argon. A hydrazine is weighed into a round bottom flask and solvent is added. The solutions described above are combined and stirred at about 80° C. for about 5 h. The crude reaction material is then transferred to new flask, suitable solvent is added along with 6 N HCl. The mixture is stirred vigorously for about 14 h. Additional solvent is added to the reaction mixture followed by portion wise addition of K₂CO₃ until the pH of the solution was about 9.5. The resulting reaction mixture is dried and concentrated.

### Exemplification of General Procedure H

### Preparation of 1-(4-((1S,3R)-3-amino-3-(hydroxymethyl)cyclopentyl)phenyl)-5-phenylpentan-1-one

All the glassware was oven dried for 4 h prior to use. DME was purged with argon for 1.5 h prior to use. A 1 L three neck flask equipped with a magnetic stirrer and J-Kem thermocouple was charged with dichloro[bis(diphenylphosphinophenyl)ether]palladium(II) [also known as DPEphos] (9.34 g, 13.05 mmol) and the three neck flask was purged with argon for about 30 min. A separate 1 L flask containing a magnetic stir bar was taken inside an inert atmosphere glove box and was charged with LHMDS (175 g, 1044 mmol). The flask was brought outside the glove box and ((1R,3S)-1-amino-3-(4-bromophenyl)cyclopentyl)methanol hydrochloride (80 g, 261 mmol) was added to the flask followed by the addition of DME (175 mL). The reaction mixture was stirred at RT for about 30 min while being purged with argon. (E)-1-tert-butyl-2-(5-phenylpentylidene)hydrazine (76 g, 326 mmol) was weighed into a 250 mL round bottom flask and DME (25 mL) was added. The solution was cannula transferred to the 1 L flask. The 250 mL flask was rinsed with DME (25 mL). The 1 L flask was further purged with argon for about 20 min and the reaction mixture was then cannula transferred to the three neck flask. The 1 L flask was rinsed with DME (50 mL) and cannula transferred to the three neck flask. The three neck flask was then maintained at positive pressure of argon ensuring there was no significant solvent loss and stirred at about 78° C. for about 5 h. Crude reaction material was then transferred to 5 L three neck flask. THF (250 mL), MeOH (250 mL) and 6 N HCl (400 mL) were added to the flask. The mixture was stirred vigorously for about 14 h. CH₂Cl₂ (200 mL) was added to the reaction mixture followed by portion wise addition of K₂CO₃ until the pH of the solution was about 9.5. 1-(4-((1S,3R)-3-amino-3-(hydroxymethyl)cyclopentyl)phenyl)-5-phenylpentan-1-one (66.2 g, 72%) was obtained.

The crude reaction mixture obtained above contained about 2-10% of the debrominated starting material (i.e., ((1R,3S)-1-amino-3-(4-phenyl)cyclopentyl)methanol hydrochloride) as a side product. The above reaction mixture in the 5 L flask was transferred to a 4 L separatory funnel and was diluted with CH₂Cl₂ (500 mL) and water (500 mL). The organic layer was separated and the aqueous layer was washed with CH₂Cl₂ (200 mL). The combined organic layer was washed thrice with water (1 L). The organic layer was concentrated in vacuo and then diluted with IPAc (500 mL). The aqueous layer was washed twice with 500 mL 5% Cysteine+10% K₂CO₃ solution. The organic layer was then washed with saturated ammonium chloride solution (500 mL), dried over Na₂SO₄ and concentrated in vacuo. 1-(4-((1S,3R)-3-amino-3-(hydroxymethyl)cyclopentyl)phenyl)-5-phenylpentan-1-one (58.9 g, 65%) was obtained. The amount of ((1R,3S)-1-amino-3-(4-phenyl)cyclopentyl)methanol hydrochloride was about 0.5 mol %. The formation of (R)-mandelic acid salt of the product lowered the amount of ((1R,3S)-1-amino-3-(4-phenyl)cyclopentyl)methanol hydrochloride to below 0.2 mol % level.

### General Procedure I and Exemplification Thereof

### Sonogashira Coupling of an α-Amino Alcohol-Containing Compound and an Alkyne

As shown in FIG. 7, an alkyne is charged slowly to a reaction mixture over about 2 h at about 65° C. The mixture was stirred at about 65° C. for about another 6 h, until HPLC shows the reaction is substantially complete.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of making a compound of formula I or a salt thereof, 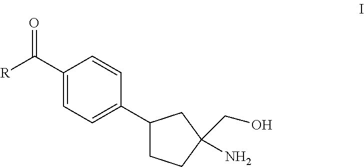
comprising the step of combining a compound of formula II or a salt thereof, 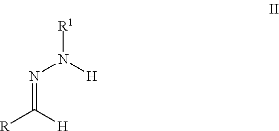
a compound of formula III or a salt thereof, 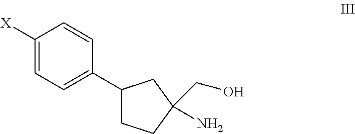
a metal catalyst, a base, and an organic solvent;
wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;
R¹ is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;
X is halogen or sulfonate; and
the molar ratio of base to the compound of formula III is greater than or equal to about 2.

2. The method of claim 1 wherein the metal catalyst comprises palladium.

3. The method of claim 1 wherein the base is a bis(trialkylsilyl)amide salt and the organic solvent is 1,4-dioxane or dimethoxyethane.

4. The method of claim 1 wherein R¹ is optionally substituted alkyl, optionally substituted cycloalkyl, optionally substituted aryl or optionally substituted heteroaryl.

5. The method of claim 1 wherein R is optionally substituted arylalkyl.

6. A method of extracting (1-amino-3-phenylcyclopentyl)methanol from a mixture comprising (1-amino-3-phenylcyclopentyl)methanol and a compound of formula I or a salt thereof, 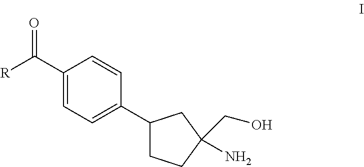
in an organic solvent, comprising the step of contacting the mixture with aqueous potassium carbonate having a pH of between about 9 and about 9.5, thereby extracting (1-amino-3-phenylcyclopentyl)methanol from the mixture;
wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

7. The method of claim 6 wherein R is optionally substituted aralkyl and the organic solvent is 1,4-dioxane or dimethoxyethane.

8. A method for preparing the (R)-mandelic salt of a compound of formula I, 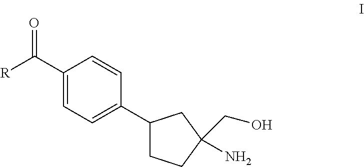
comprising the step of combining (R)-mandelic acid and a compound of formula I in an organic solvent, thereby forming the (R)-mandelic salt of a compound of formula I; wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

9. The method of claim 8 wherein the organic solvent is 1,4-dioxane or dimethoxyethane and R is optionally substituted aralkyl.

10. A method of making a compound of formula IV or a salt thereof: 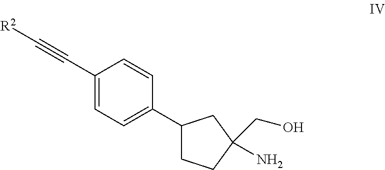
comprising the step of combining a compound of formula III or a salt thereof, 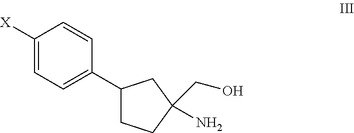
a compound of formula V: 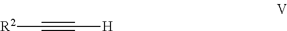
a metal catalyst, and an organic solvent; wherein,
R² is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl; and
X is halogen.

11. The method of claim 10 wherein the metal catalyst comprises palladium.

12. The method of claim 10, wherein the organic solvent is 1,4-dioxane or dimethoxyethane and R² is alkoxy-substituted alkyl.

13. A method of making a compound of formula III or a salt thereof, 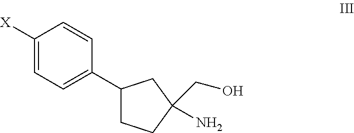
comprising the step of combining a compound of formula VI or a salt thereof: 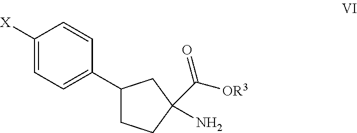
and a reducing agent; wherein,
X is halogen or sulfonate; and
R³ is alkyl.

14. A method of making a compound of formula IA or a salt thereof, 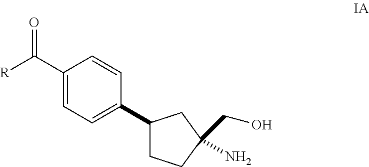
comprising the step of combining a compound of formula II or a salt thereof, 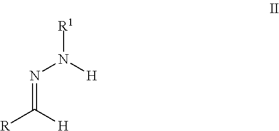
a compound of formula III or a salt thereof, 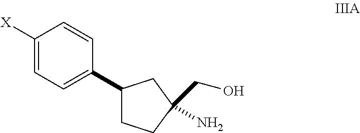
a metal catalyst, a base, and an organic solvent; wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;
R¹ is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl;
X is halogen or sulfonate; and
the molar ratio of base to the compound of formula IIIA is greater than or equal to about 2.

15. The method of claim 14, wherein the metal catalyst comprises palladium.

16. The method of claim 14 wherein the base is a bis(trialkylsilyl)amide salt and the solvent is 1,4-dioxane or dimethoxyethane.

17. The method of claim 14, wherein R¹ is alkyl, substituted alkyl, aryl or heteroaryl.

18. The method of claim 14 wherein R is optionally substituted aralkyl.

19. A method of extracting ((1R,3R)-1-amino-3-phenylcyclopentyl)methanol from a mixture comprising ((1R,3R)-1-amino-3-phenylcyclopentyl)methanol and a compound of formula IA or a salt thereof, 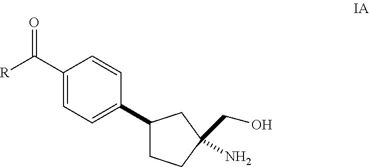
in organic solvent, comprising the step of contacting the mixture with aqueous potassium carbonate having a pH of between about 9 and about 9.5, thereby extracting ((1R,3R)-1-amino-3-phenylcyclopentyl)methanol from the mixture; wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

20. The method of claim 19 wherein R is optionally substituted aralkyl and the organic solvent is 1,4-dioxane or dimethoxyethane.

21. A method of preparing the (R)-mandelic salt of a compound of formula IA, 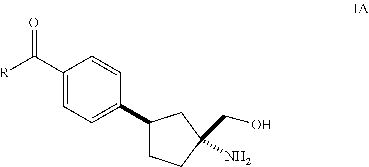
comprising the step of adding (R)-mandelic acid to a compound of formula IA or salt thereof in an organic solvent, thereby forming the (R)-mandelic salt of the compound of formula IA; wherein,
R is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

22. The method of claim 21 wherein R is optionally substituted aralkyl and the organic solvent is 1,4-dioxane or dimethoxyethane.

23. A method of making a compound of formula IVA or a salt thereof: 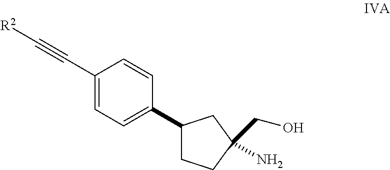
comprising the step of combining a compound of formula IIIA or a salt thereof, as defined above, a compound of formula V: 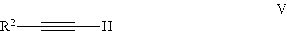
a metal catalyst, and an organic solvent; wherein,
R² is optionally substituted alkyl, optionally substituted cycloalkyl, optionally substituted aryl, optionally substituted heteroaryl, optionally substituted heterocyclyl, optionally substituted aralkyl, optionally substituted heteroaralkyl, optionally substituted cycloalkylalkyl, or optionally substituted heterocyclylalkyl.

24. The method of claim 23 wherein the metal catalyst comprises palladium.

25. The method of claim 23 wherein the organic solvent is 1,4-dioxane or dimethoxyethane and R² is alkoxy-substituted alkyl.

26. A method of making a compound of formula IIIA or a salt thereof, 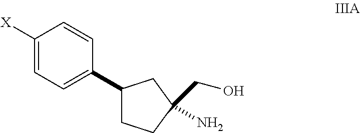
comprising the step of combining a compound of formula VIA or a salt thereof: 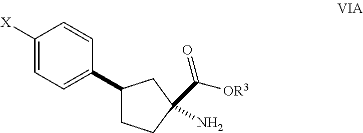
and a reducing agent; wherein,
X is halogen or sulfonate; and
R³ is alkyl.
